(19) United States
(12) Patent Application Publication
(10) Pub. No.: US 2015/0094794 A1
(43) Pub. Date: Apr. 2, 2015

(54) WIRE COLLECTION DEVICE WITH INCREASING COLLECTION DIAMETER

(71) Applicant: Cook Medical Technologies LLC, Bloomington, IN (US)

(72) Inventors: Sean Cummins, Mungret (IE); Kieran Costello, Ballina/Killaloe (IE); Patrick Kelly, Clarina (IE)

(21) Appl. No.: 14/489,219

(22) Filed: Sep. 17, 2014

Related U.S. Application Data
(60) Provisional application No. 61/885,330, filed on Oct. 1, 2013.

(51) Int. Cl. A61F 2/966

(52) U.S. Cl. CPC A61F 2/966
USPC 623/1.11

(57) ABSTRACT

A stent deployment system includes a wire collection device with a profiled retraction wire and an actuator coupled to a collection spindle. A distal end of the profiled retraction wire is coupled to a sledge that is coupled to an outer sheath. A proximal end of the profiled retraction wire is coupled to the collection spindle. The actuator can actuate rotation of the collection spindle to collect the profiled retraction wire around the collection spindle, and collection of the profiled retraction wire retracts the outer sheath across an outer surface of the stent so as to deploy a stent surrounded by a distal end of the outer sheath. The wire collection device has a varying collection diameter that includes the diameter of the collection spindle and the combined thickness of collected portions of the profiled retraction wire.

### CROSS-REFERENCE TO RELATED APPLICATIONS

This application is a non-provisional application which claims priority to U.S. provisional application Ser. No. 61/885,330, filed Oct. 1, 2013, which is incorporated by reference herein in its entirety.

### BACKGROUND

Current delivery systems for self-expanding stents generally employ “pin and pull” systems that include an inner catheter extending through an outer sheath. Typically, the stent is placed inside the outer sheath and held in a compressed position by the outer sheath as the outer sheath and inner catheter are inserted into a patient's body vessel. To deploy the stent, the user retracts, or pulls, the outer sheath using one hand while the other hand holds the inner catheter stationary to maintain position of the stent as the outer sheath is retracted, thereby allowing the stent to gradually expand as the outer sheath uncovers the stent.

In these “pin and pull” systems, the user has difficulty maintaining the position of the inner catheter while pulling on the outer sheath because of resistance between the inner catheter and outer sheath, between the outer sheath and the stent, and between the outer sheath and the surrounding vascular walls, or other surrounding blood vessel or body vessel. To overcome this resistance the user may need to exert a large amount of force that leads to various complications, including for example, inaccurate stent positioning, displacement of the stent, shortening or lengthening of the stent, or other damage to the structure of the stent, or damage to the target vessel.

“Pin and pull” systems may also have other disadvantages, including, for example, lack of control during stent deployment and requirement of assistance from a second person. The resistance between the outer sheath and stent varies as more of the stent is uncovered and the stent expands. Specifically, the stent's self-expanding outward circumferential bias frictionally binds it against the outer sheath. During sheath retraction, this binding force decreases as the stent is released, which correspondingly decreases the retraction force needed on the outer sheath. Thus, stent deployment may be difficult to control because the required deployment force varies as the outer sheath retracts across the surface of the stent. As a result, the user must vary the force applied to the outer sheath and the inner catheter in order to maintain a steady deployment speed and ensure accurate stent placement. In most pin and pull systems, the ratio of handle movement to stent deployment distance is 1:1, requiring the user to move faster to deploy longer stents and increasing difficulty in controlling the stent. Because the user's hands are holding the distal ends of the outer sheath and inner catheter, the user cannot easily monitor or attend to the positioning of the outer sheath in the hemostasis valve to ensure accurate stent placement, such that an assistant must be present to attend to the positioning of the outer sheath in the hemostasis valve and accurate positioning of the stent.

Other vascular stent placement delivery systems offer one-handed operation by converting hand-movements into indexed movement of the outer sheath. Such systems generally still operate, however, with a 1:1 ratio of handle movement to stent deployment distance. In other words, such systems do not provide mechanical advantage to accommodate, or reduce the amount of work required for, deployment of longer stents as compared to deployment of shorter stents.

### BRIEF SUMMARY

In one aspect, a stent deployment system includes a wire collection device with a profiled retraction wire and an actuator coupled to a collection spindle. A distal end of the profiled retraction wire is coupled to a sledge that is coupled to a proximal end of an outer sheath, and a distal end of the outer sheath surrounds a stent. A proximal end of the profiled retraction wire is coupled to the collection spindle. The actuator can actuate rotation of the collection spindle to collect the profiled retraction wire around the collection spindle, and collection of the profiled retraction wire retracts the outer sheath across an outer surface of the stent so as to deploy the stent. The wire collection device has a varying collection diameter that includes the diameter of the collection spindle and the combined thickness of collected portions of the profiled retraction wire.

In another aspect, a stent deployment system includes a wire collection device with a profiled retraction wire and a thumbwheel coupled to a collection spindle. The profiled retraction wire is coupled to an outer sheath to retract the outer sheath. Retracting the outer sheath deploys a stent from a distal end of the outer sheath. The thickness of the profiled retraction wire increases from its proximal end to towards its distal end. The proximal end of the profiled retraction wire is attached to the collection spindle and the distal end of the profiled retraction wire is coupled to a proximal end of the outer sheath. The thumbwheel can actuate rotation of the collection spindle to collect the profiled retraction wire around a collection diameter, which includes the diameter of the collection spindle and the thickness of the retraction wire. As the profiled retraction wire is collected around the collection spindle, the collection diameter increases.

### BRIEF DESCRIPTION OF THE DRAWINGS

FIG. 1A is a view of an exemplary wire collection device for a stent delivery system;

FIG. 1B is a diagrammatic cross-sectional illustration of a detail/partial view of an exemplary wire collection device for a stent delivery system;

FIG. 2A is a diagrammatic longitudinal cross-sectional illustration of an exemplary wire collection device for a stent delivery system;

FIG. 2B is a diagrammatic longitudinal cross-sectional illustration of a partial view of an exemplary wire collection device for a stent delivery system;

FIG. 3 is a diagrammatic longitudinal cross sectional illustration of a partial view of an exemplary wire collection device for a stent delivery system;

FIG. 4 is a diagrammatic longitudinal cross sectional illustration of a partial view of an exemplary wire collection device for a stent delivery system;

FIG. 5A is a diagrammatic longitudinal cross sectional illustration of exemplary wire collection device for a stent delivery system;

FIG. 5B is a diagrammatic longitudinal cross sectional illustration of a partial view of an exemplary wire collection device for a stent delivery system;

FIG. 5C is a diagrammatic longitudinal cross sectional illustration of a partial view of an exemplary wire collection device for a stent delivery system;

FIG. 6A is a diagrammatic longitudinal cross sectional illustration of exemplary wire collection device for a stent delivery system;

FIG. 6B is a diagrammatic longitudinal cross sectional illustration of a partial view of an exemplary wire collection device for a stent delivery system;

FIG. 7A is a diagrammatic longitudinal cross sectional illustration of exemplary wire collection device for a stent delivery system;

FIG. 7B is a diagrammatic longitudinal cross sectional illustration of exemplary wire collection device for a stent delivery system;

FIG. 8A is a diagrammatic longitudinal cross sectional illustration of a partial view of an exemplary wire collection device for a stent delivery system;

FIG. 8B is a diagrammatic longitudinal cross sectional illustration of a partial view of an exemplary wire collection device for a stent delivery system;

FIG. 8C is a diagrammatic longitudinal cross sectional illustration of a partial view of an exemplary wire collection device for a stent delivery system;

FIG. 9 is a required deployment force profile showing variation in required deployment force relative to stent deployment distance for a stent delivery system; and

FIG. 10 is an applied force profile of a force variation curve for an exemplary wire collection device for a stent delivery system.

### DETAILED DESCRIPTION

Various embodiments are described below with reference to the drawings. The relationship and functioning of the various elements of the embodiments may better be understood by reference to the following detailed description. However, embodiments are not limited to those illustrated in the drawings. It should be understood that the drawings are not necessarily to scale, and in certain instances details may have been omitted that are not necessary for an understanding of embodiments disclosed herein, such as—for example—conventional fabrication and assembly. The invention is defined by the claims, may be embodied in many different forms, and should not be construed as limited to the embodiments set forth herein; rather, these embodiments are provided so that this disclosure will be thorough and complete, and will fully convey enabling disclosure to those skilled in the art. As used in this specification and the claims, the singular forms “a,” “an,” and “the” include plural referents unless the context clearly dictates otherwise.

A wire collection device for a stent delivery system is provided in some embodiments. The stent delivery system includes a profiled retraction wire that is attached at a proximal end to a wire collection device, which is rotatable to pull and collect the retraction wire around a wire collection spindle. The wire collection device includes a thumbwheel, or other actuator, coupled to the wire collection spindle. Retraction of the outer sheath is controlled by turning the thumbwheel, or activating the actuator, to actuate rotation of the collection spindle to pull and collect the retraction wire around the collection diameter. The collection diameter increases as the retraction wire collects around the spindle, and as the profile of the retraction wire changes. The wire collection device may further include a diameter increasing construct that may be attached to the retraction wire or to the collection spindle. The system may include one or more retraction wires connected by a distal end to the proximal end of the outer sheath, and connected by a proximal end to the wire collection spindle, so that rotation of the collection spindle collects the one or more retraction wires to retract the outer sheath.

A distal end of the retraction wire is connected or attached to a proximal end of an outer sheath that holds a stent at or near a distal end of an inner catheter. The inner catheter extends through the outer sheath from a proximal end near the wire collection device to a distal end near the stent. The stent and the distal ends of the inner catheter and outer sheath are inserted into a body vessel until the stent is located at a desired location. As the thumbwheel is turned, or actuator is activated, the wire collection device pulls and collects the retraction wire around the collection spindle, thereby retracting the outer sheath across the inner catheter to uncover the stent while the internal catheter holds the stent in the desired location. The stent may be a self-expanding stent, or a stent that is expanded by the force of a balloon.

The terms “outer sheath” and “inner catheter,” as used herein, mean a tube, cannula, or other similar structure, that may have solid, woven, braided, porous, smooth, or other type of wall with one or more apertures extending through all or part of the structure. As used herein, “retraction wire” means a rope, cord, wire, cable, belt, chain, or any other strand(s) of material that is suitable for use in a stent deliver system to retract or pull an outer sheath, or cannula, to allow stent deployment or delivery. The term “collection spindle,” as used herein, means one or more axles, spindles, or generally cylindrical structures around which the retraction wire is wound or collected. The wire collection device may be used with one or more retraction wires, and may include one or more collection spindles. As used herein, “collection diameter” refers to the diameter around which a retraction wire collects, or is wound. Thus, the “collection diameter” may increase as the retraction wire overlaps itself as it is being collected, or wound, around an axle, spindle, collection drum, or other cylindrical structure.

When the wire collection device is used with a self-expanding stent, the required deployment force may be greater during initial deployment of the stent and may decrease as the outer sheath, or cannula, begins to move and/or uncovers more of the stent. As used herein, “required deployment force” refers to an amount of force required to overcome the frictional forces (e.g., static friction) between the outer sheath and the stent, frictional forces between the outer sheath and an inner catheter, or cannula, that holds the stent in place as the outer sheath is retracted, and frictional forces between the outer sheath and the surrounding body vessels where the stent is being implanted or placed.

The wire collection device may provide the user of the stent delivery system with a more consistent “touch and feel” by reducing the variation in applied force, or the amount of force applied by the user, to deploy the stent. As used herein, the term “applied force” means the amount of force applied to the wire collection device by the user. For example, the applied force may be the amount of force applied to turn the thumbwheel and generate the required deployment force. Reducing variation in the required applied force may be accomplished by varying the mechanical advantage provided to the user as the required stent deployment force increases, where the mechanical advantage increases and the collection diameter increases. The collection spindle and profiled collection wire may be sized and configured to control the degree and rate of change in the mechanical advantage provided during stent deployment.

The wire collection device may provide a mechanical advantage such that the deployment distance, or retraction distance of the outer sheath, increases with hand movements of the user, or revolutions of the thumbwheel. The wire collection device controls retraction of the outer sheath so as to improve user feel and control for accurately positioning the stent. The wire collection device may be configured so that the user may exert a steady, or consistent force, throughout the deployment despite variation in the force required to retract the outer sheath, or deploy the stent. The wire collection device may provide a mechanical advantage to the user that results in a 1:1 ratio, or greater than or less than a 1:1 ratio, of handle movement to stent deployment distance.

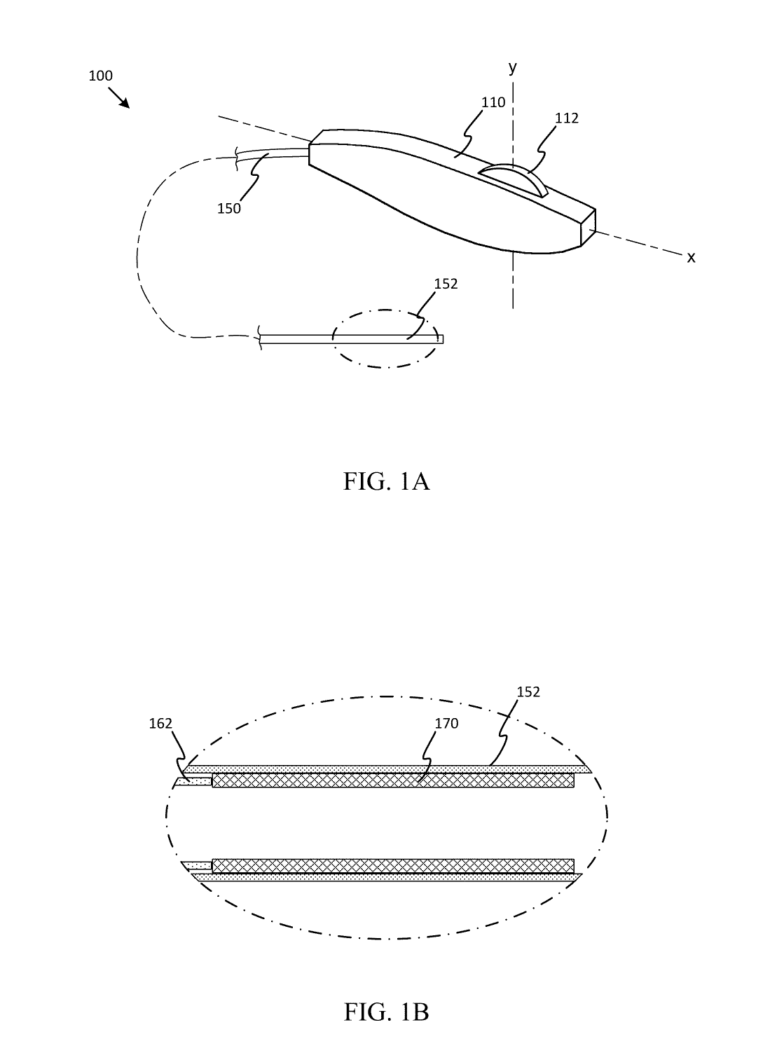

In some embodiments, as illustrated with reference to FIGS. 1A-B and 2A-B, a stent delivery system 100 includes a profiled retraction wire 102 coupled to an outer sheath 104, an inner catheter 106 extending through the outer sheath 104, a wire collection device 108, and a handle 110 housing the wire collection device 108. For example, the handle 110 may have a length of about 100 mm and a height of about 40 mm-50 mm. The inner catheter 106 is shown truncated to maintain clear illustration of the thumbwheel 112, but in different embodiments, its proximal end will generally protrude through the housing of the handle 110 and may include a luer structure for ease of attaching a fluid-delivery device (e.g., for delivering flushing fluid, radio-opaque contrast fluid, or other fluid), and it may also serve as a passage for a wire guide. A distal end 140 of the profiled retraction wire 102 may be attached, directly or indirectly, to a proximal end 150 of the outer sheath 104 and at or near a proximal end 160 of the inner catheter 106. The proximal end 142 of the retraction wire is attached to the collection spindle 114. The proximal end 160 of the inner catheter 104 is attached to the handle 110. The distal end 152 of the outer sheath 104 retractably surrounds a stent 170 located at or near a distal end 162 of the inner catheter 106. Sheath retraction may be monitored, for example, by fluoroscopy, or with the use of radiopaque markers placed on the outer sheath 104 and inner catheter that align when retraction is complete. Alternatively, a lock or other mechanism may be configured to stop rotation of the thumbwheel 112 after a certain length of the retraction wire 102 has been collected.

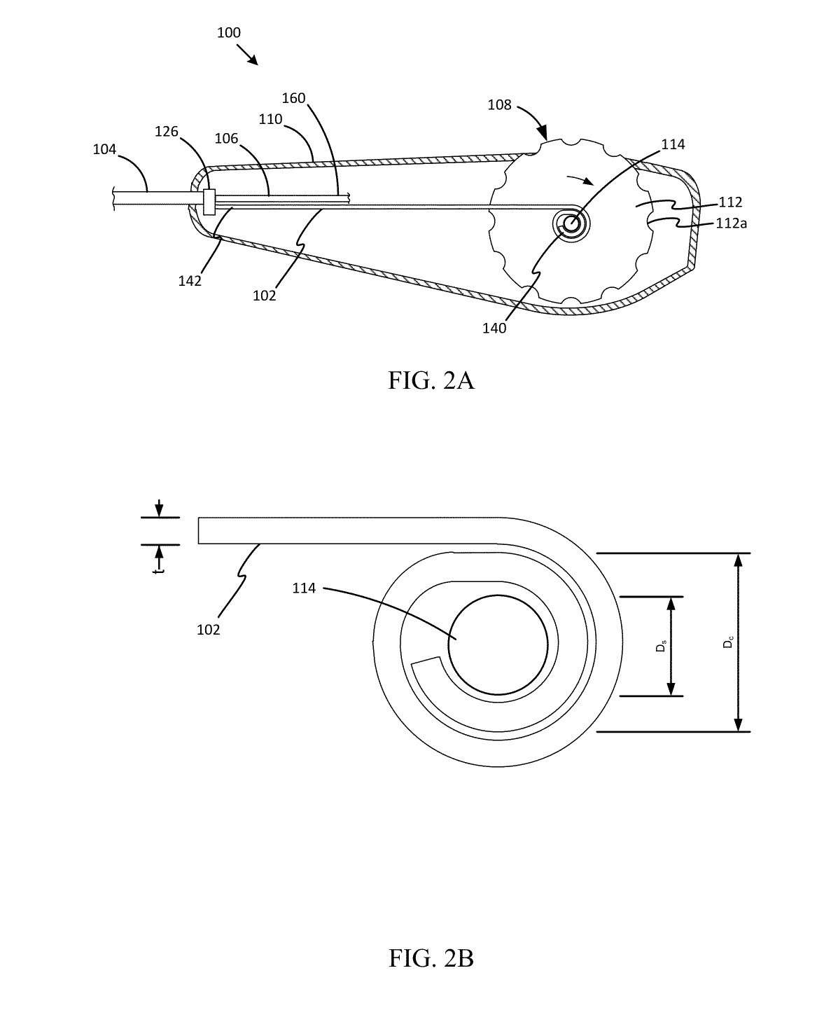

With reference to FIG. 2A, the wire collection device 108 includes a thumbwheel 112 mounted on a collection spindle 114. The profiled retraction wire 102 has a substantially constant profile with a thickness t along the length of the wire 102. Alternatively, the retraction wire 102 may have a profile that varies along the retraction wire 102. For example, the retraction wire 102 may have a thickness that increases or decreases, at a linear or curvilinear rate or in increments, from the proximal end 140 towards the distal end 142 of the retraction wire 102. The collection diameter D_{c} of the wire collection device 108 increases with each full revolution of the collection spindle 114 as the retraction wire 102 collects around the collection spindle 114 and overlaps itself. The thumbwheel 112 may include notches 112a to provide better grip to the user. Alternatively, the thumbwheel 112 may have a rough, gritty, or cross-hatched contact surface, or may be covered by or made of material, such as rubber or silicon, that provide traction to the user. For example, the thumbwheel 112 may be formed by a two shot mold process, and/or may include materials containing acetyl or acrylonitrile butadiene styrene (ABS). The wire collection device 108 increases the mechanical advantage provided to the user as the collection diameter 118 increases. Therefore, the ratio of movement of the thumbwheel 112 to stent deployment distance decreases as the collection diameter increases. This may provide the user with a more consistent “feel” throughout the deployment of the stent. The features in this and other presently disclosed embodiments may provide significantly improved control and precision over other wire-winding structures.

In some embodiments, the wire collection device 108 may also include a ratchet that allows the thumbwheel 112 to rotate in one direction and prevents rotation in the opposite direction. For example, when the user releases the thumbwheel 112, the ratchet may prevent the retraction wire 102 from unwinding from the spindle 114. The ratchet may be a pawl and gear ratchet located on or coupled to the thumbwheel 112. Alternatively, the ratchet may act directly on the retraction wire 102, for example, as with a cable tie or tie wrap. In some embodiments, the ratchet may have a high friction surface that acts on the thumbwheel 112 to allow the thumbwheel 112 to move in one direction and prevent the thumbwheel 112 from rotating in the opposite direction. The thumbwheel 112 and collection spindle 114 may be made of rubber, plastic, metal, or other material that is sufficiently rigid to withstand the force required to turn the thumbwheel 112 and the required deployment force, and sufficiently lightweight for use in a surgical procedure.

With reference to FIG. 2B, the collection diameter D_{c} is approximately equal to the sum of the diameter Dₛ of the collection spindle 114 and the combined thicknesses t of layers of the profiled retraction wire 102 that are collected around the collection spindle 114. For example, after the first full revolution of the collection spindle 114, the collection diameter D_{c} is approximately:

D_{c}=Dₛ+2(t)

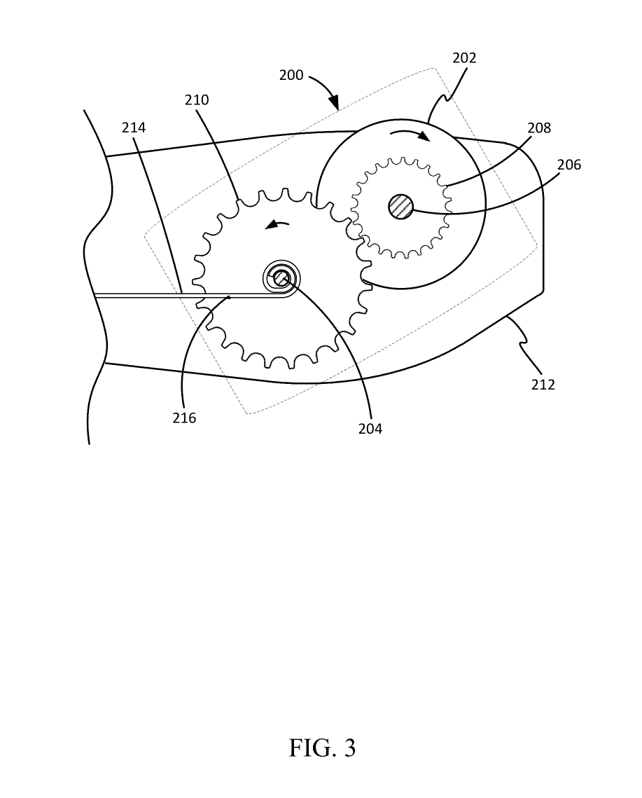

In some embodiments, as illustrated with reference to FIG. 3, the thumbwheel of a wire collection device 200 for a stent delivery system has a thumbwheel 202 that may be offset from the collection spindle 204. For example, the thumbwheel 202 may be mounted on an axle 206 that is axially offset from the collection spindle 204. The thumbwheel 202 is coupled to the collection spindle 204 for example, by a transmission mechanism, such as mesh gears 208, 210. Mesh gear 208 may be formed or molded as part of the thumbwheel 202, or may be a separate component that is mounted on the axle 206 adjacent or next to the thumbwheel 202. Mesh gear 210 may be formed or molded as part of the spindle 204, or may be a separate component that is co-axial with the spindle 204. The spindle 204 may have a diameter that is larger or smaller than the diameter of the thumbwheel 202. The thumbwheel 202, spindle 204, axle 206, and mesh gears 208, 210 are sized to be housed in handle 212, through which a portion of the thumbwheel 202 protrudes to allow the user to turn the thumbwheel 202. When the thumbwheel 202 rotates, gear 208 engages gear 210, thereby actuating rotation of the collection spindle 204 to collect the retraction wire 214 around the spindle 204. In other embodiments, the transmission mechanism may include a transmission belt, a rack and pinion, a clutch, a ratchet, or any combination thereof.

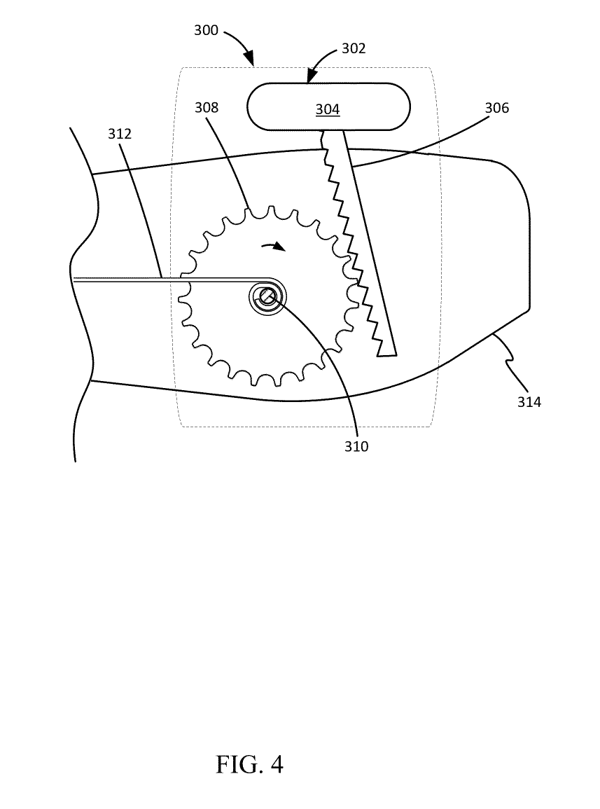

In some embodiments, as illustrated with reference to FIG. 4, the wire collection device 300 has an actuator 302 that includes a push button 304 and a rod, or post, 306, or a push button rod, that is configured to engage gear 308. The rod 306 and gear 308 form a rack and pinion that actuates rotation of the collection spindle 310 to rotate and collect the profiled retraction wire 312 around the collection spindle 310. The rod 306, gear 308, and spindle 310 are sized to be housed in handle 314, through which a portion of the rod 306 protrudes to attach to push button 304. When the push button 304 is pressed, the rod 306 engages the gear 308 to rotate the collection spindle 310 in one direction. When the push button 304 is released, the rod 306 may act as a ratchet to prevent rotation of the spindle 310 in the opposite direction, so as to prevent the retraction wire 312 from unwinding. The rod 306 may be configured so that one full depression of the actuator 302 is sufficient to complete stent deployment. Alternatively, after depression, the actuator 302 may spring back to its initial position, and subsequent depression actuates further rotation of the collection spindle 310 until sheath retraction, or stent deployment, is complete. One of skill in the art would recognize that other actuating mechanisms may be used while keeping within the spirit and scope of the embodiments illustrated herein.

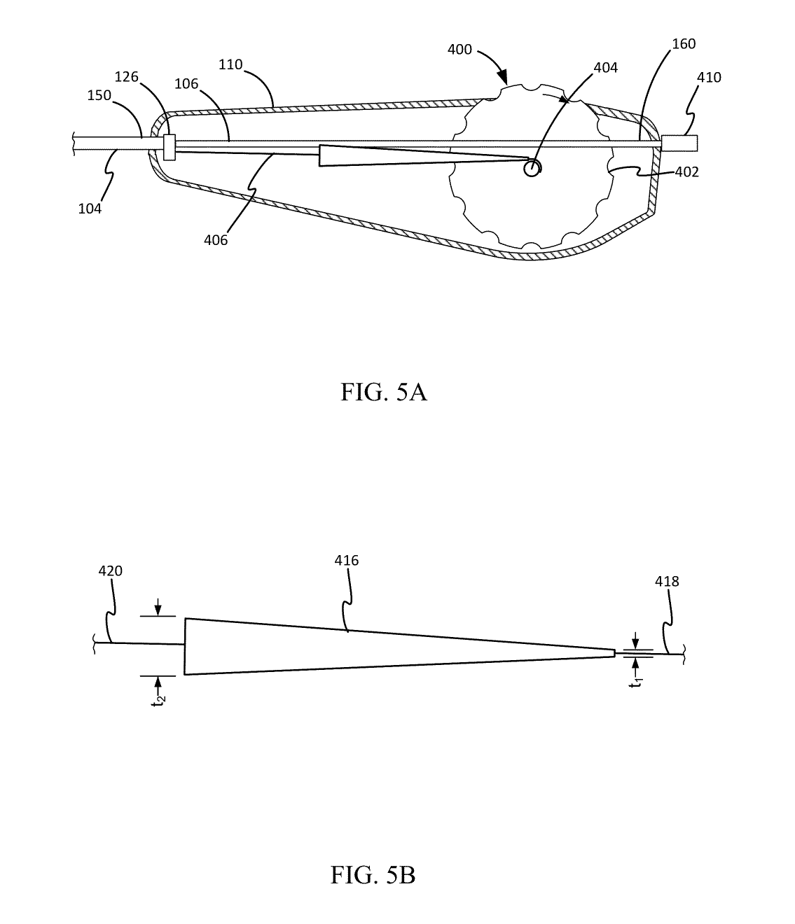

In some embodiments, as illustrated with reference to FIGS. 5A-C, the wire collection device 400 for a stent deliver system includes a thumbwheel 402, a collection spindle 404, and a profiled retraction wire 406. A distal end 408 of the retraction wire 406 is attached by a sledge 126 to a proximal end 150 of the outer sheath 104. Alternatively, the retraction wire 406 may be attached to the outer sheath 104 directly, such as, for example, by embedding the retraction wire 406 in walls of the outer sheath 104, or welding the retraction wire 406 to the outer sheath 104. An inner catheter 106 extends through an aperture of the outer sheath 104. A proximal end 160 of the inner catheter 106 is attached to the handle 110 that houses the thumbwheel 402, collection spindle 404, and retraction wire 406. The proximal end 160 of inner catheter 106 protrudes through the housing of the handle 110 and may include a luer structure 410 for ease of attaching a fluid-delivery device (e.g., for delivering flushing fluid, radio-opaque contrast fluid, or other fluid), and it may also serve as a passage for a wire guide.

Rotation of the thumbwheel 402 actuates rotation of the collection spindle 404 to collect the retraction wire 406 around the collection spindle 404. The collection diameter of the retraction wire 406 increases as the collection spindle 404 rotates and the retraction wire 406 collects around itself. The profile of the retraction wire 406 may increase gradually from the proximal end 414 of the retraction wire 406, which is attached to the collection spindle 404, towards the distal end 416 of the retraction wire 406, which is attached, directly or indirectly, to the proximal end 150 of the outer sheath 104. For example, the thickness of the profiled retraction wire 406 may increase linearly or curvilinearly along a portion 416 of the retraction wire 406 from a first thickness t₁, nearer the proximal end 418 of the retraction wire 406, to a second thickness t₂, nearer the distal end 420 of the retraction wire 406. Alternatively, the thickness of the profiled retraction wire 406 may decrease from the proximal end 418 towards the distal end 420 of the retraction wire 406. The portion 416 of the retraction wire 406 having a varying profile may be a diameter increasing construct that is attached to the retraction wire 406, or may be formed as a unitary piece with the retraction wire 406.

In some embodiments, as illustrated with reference to FIG. 5C, the retraction wire 406 may have a varying profile 416a that has a constant profile along a first portion 416b and a varying profile or thickness along a second portion 416c. For example, the first portion 416b may have a substantially constant thickness t₃, and the second portion 416c may have thickness that varies from a smaller thickness t₄ to a larger thickness t₅. The first portion 416b may be nearer the proximal end 418 of the profiled retraction wire 406, and the second portion 416c may be nearer the distal end 420 of the profiled retraction wire 406. Alternatively, the first portion 416b may be nearer the distal end 420 and the second portion may be nearer the proximal end 418. The first and second portions 416b, 416c may be diameter increasing constructs that are attached to the retraction wire 406, or may be formed as a unitary piece with the retraction wire 406. The profile or thickness of the retraction wire 406 may vary with the required deployment force, and may be configured to provide the user with a more consistent “touch and feel” by reducing the variation in amount of force required from the user to deploy the stent. For example, by configuring the rate of change in the thickness of the profiled retraction wire 406, the mechanical advantage provided to the user as the required stent deployment force may be adjusted to account for variation in the required deployment force.

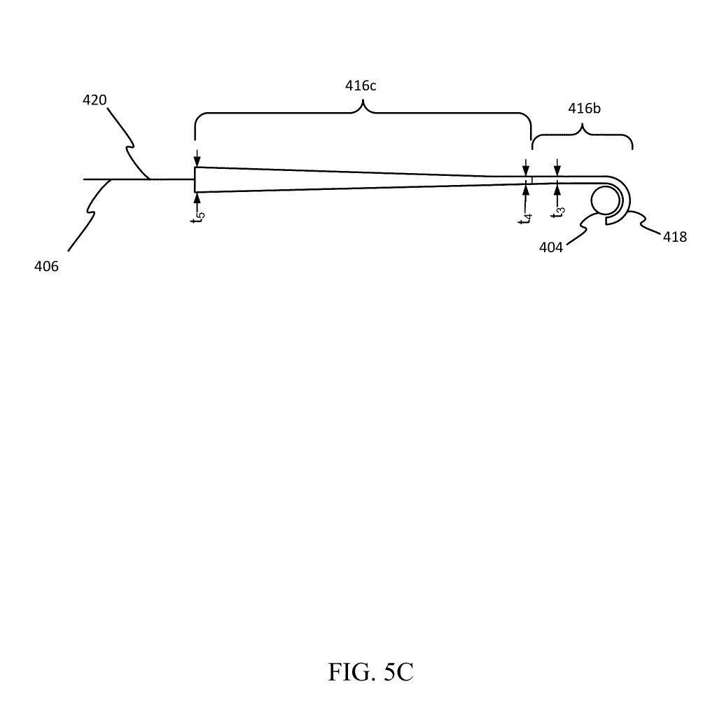

In some embodiments, as illustrated with reference to FIGS. 6A-B, the wire collection device 500 has a thumbwheel 502, a collection spindle 504, and a profiled retraction wire 506 with a distal end 508 coupled to the proximal end 150 of the outer sheath 104. For example, the distal end 508 of the retraction wire may be coupled to the outer sheath 104 by a sledge 126. An inner catheter 106 extends through an aperture of the outer sheath 104. The proximal end 160 of inner catheter 106 protrudes through the housing of the handle 110 and may include a luer structure 510 for ease of attaching a fluid-delivery device (e.g., for delivering flushing fluid, radio-opaque contrast fluid, or other fluid), and it may also serve as a passage for a wire guide. The proximal end 512 of the retraction wire 506 is attached to the collection spindle 504.

The collection spindle 504 may include a diameter increasing construct 514, such as a cam. As the collection spindle 504 rotates, the retraction wire 506 collects around the surface of the diameter increasing construct, or cam, 514. The cam may be any shape, or type, of cam, such as, for example, an eccentric, egg-shaped, or ellipsoid cam, or may be an irregular shape. The cam 514 rotates about an off-centered point so that as the collection spindle 504 rotates, the collection diameter D_{c} increases or decreases. The cam 514 may be sized and shaped so that retraction is completed after a half revolution of the cam and so that the collection diameter D_{c} increases from a minimum to a maximum to accommodate the required deployment force. Alternatively, the cam 514 may be configured so that retraction is complete after one or more revolutions of the cam 514, such that, for example, the collection diameter D_{c} cycles from a minimum to a maximum, or increases and decreases as the cam 514 rotates.

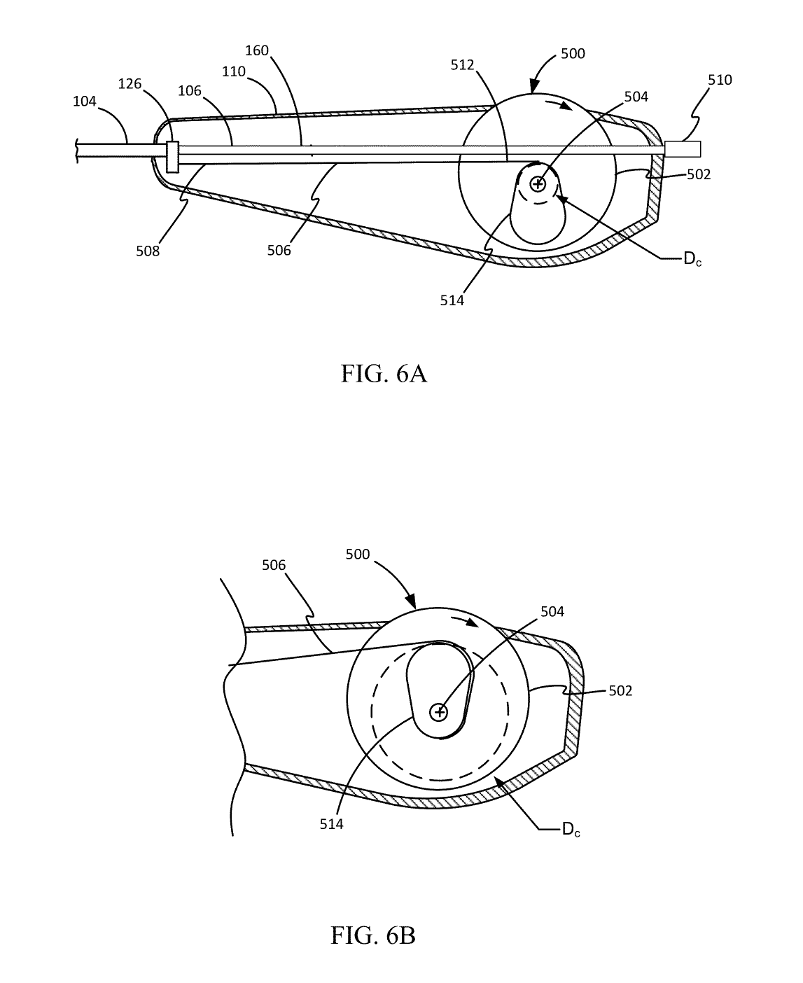

In some embodiments, as illustrated with reference to FIGS. 7A-B, the wire collection device 600 includes an actuator 602, a collection spindle 604, and a profiled retraction wire 606 with diameter increasing constructs 608. The distal end 610 of the retraction wire 606 is attached to the proximal end 150 of the outer sheath 104, for example, by a sledge 126. Alternatively, the retraction wire 406 may be attached to the outer sheath 104 directly, such as, for example, by embedding the retraction wire 606 in walls of the outer sheath 104, or welding the retraction wire 606 to the outer sheath 104. The proximal end 612 of the retraction wire 606 is attached to the spindle 304. An inner catheter 106 extends through an aperture of the outer sheath 104. A proximal end 160 of the inner catheter 106 is attached to the handle 110 that houses the actuator 602, collection spindle 604, and retraction wire 606. The proximal end 160 of inner catheter 106 protrudes through the housing of the handle 110 and may include a luer structure 614 for ease of attaching a fluid-delivery device (e.g., for delivering flushing fluid, radio-opaque contrast fluid, or other fluid), and it may also serve as a passage for a wire guide.

The actuator 602 may be a thumbwheel that, when turned, actuates rotation of the collection spindle 604 to collect the retraction wire 606 around the spindle 604. The diameter increasing constructs 608 may be sized to increase the collection diameter of the wire collection device 600, such that the collection diameter increases at a rate that accommodates the change in required deployment force. The retraction wire 606 may be positioned relative to the inner catheter 106 so that the diameter increasing constructs 608 provide support to the inner catheter 106 during sheath retraction. For example, the inner catheter 106 may rest on the diameter increasing constructs 608. The thumbwheel 602 may be aligned with the spindle 604, or may be offset from the spindle 604.

In different embodiments, for example with reference to FIG. 7B, the actuator 600a may be a push button rod that includes a push button 602a and a rod or post 602b. A gear 602c is coupled to the spindle 604. The rod 602b, gear 602c and spindle 604 are sized to be housed in handle 110, through which a portion of the rod 602b extends to attach to the push button 602a. When the push button 602a is pressed, the rod 602b engages the gear 602c to rotate the collection spindle 604 in one direction. When the push button 602a is released, the rod 602b may act as a ratchet to prevent rotation of the spindle 604 in the opposite direction, so as to prevent the retraction wire 606 from unwinding. The rod 602b may be configured so that one full depression of the actuator 600a is sufficient to complete stent deployment. Alternatively, after depression, the actuator 600a may spring back to its initial position, and subsequent depression actuates further rotation of the collection spindle 604 until sheath retraction, or stent deployment, is complete. One of skill in the art would recognize that other actuating mechanisms may be used while keeping within the spirit and scope of the embodiments illustrated herein.

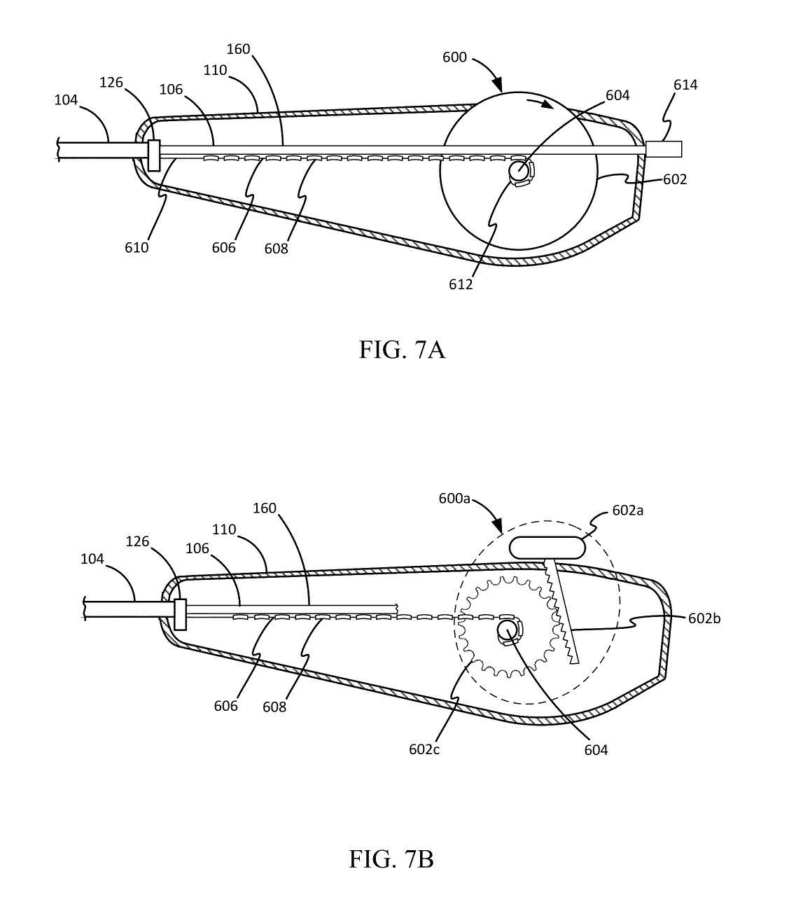

In different embodiments, as illustrated with reference to FIGS. 8A-C, the diameter increasing constructs 608a, 608b, and 608c may be configured in different sizes, shapes, and locations along the retraction wire 606. For example, with reference to FIG. 8A, the diameter increasing constructs 608a may have a groove 700 along part or all of a surface 702 of the construct 608a. The groove 700 may be sized to fit or support the inner catheter 106. The diameter increasing constructs 608a may be spaced apart along the retraction wire 606, or may be adjacent to other diameter increasing constructs 608a. As another example, with reference to FIG. 8B, the diameter increasing constructs 608b may vary in diameter, thickness, size, and/or shape along the retraction wire 606. The diameter increasing constructs 608b may increase along the retraction wire 606 from a smaller thickness t₆ nearer the spindle 604 to a larger thickness t₇ nearer the outer sheath. Alternatively, the diameter increasing constructs 608b may decrease in size from a larger thickness nearer the spindle to a smaller thickness nearer the outer sheath. The size or thickness of the diameter increasing constructs 608b may increase at a linear or non-linear rate to accommodate variation in required deployment force. The constructs 608b may be rectangular, curved, round, elliptical, or any other shape. In some embodiments, with reference to FIG. 8C, the diameter increasing constructs 608c may be a plurality of links that form the retraction wire 606. In different embodiments, the diameter increasing constructs 608a, 608b, and 608c may be used in any combination.

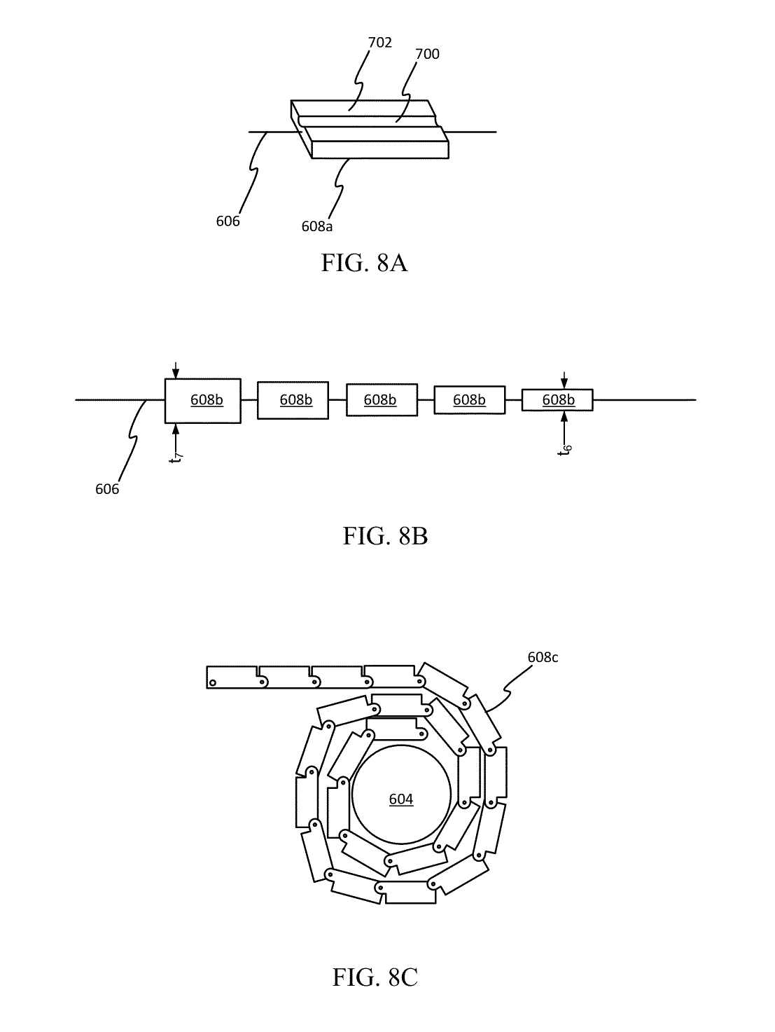

In some embodiments, for example, with reference to FIG. 9, a required deployment force profile 800 shows the required stent deployment force relative to the stent deployment distance (e.g., the distance of sheath retraction or length of retraction wire collected). The required deployment force increases from an initial force 802 to a threshold force 804, and decreases from the threshold force 804 to a lower force 806. The increase in required deployment force from the initial force 802 to the threshold force 804 may be the amount of force required to overcome the static friction and/or binding forces between a self-expanding stent and the outer sheath. Once the outer sheath begins to move, or retract, the required deployment force decreases from the threshold force 804 to the lower force 806, at the completion of sheath retraction.

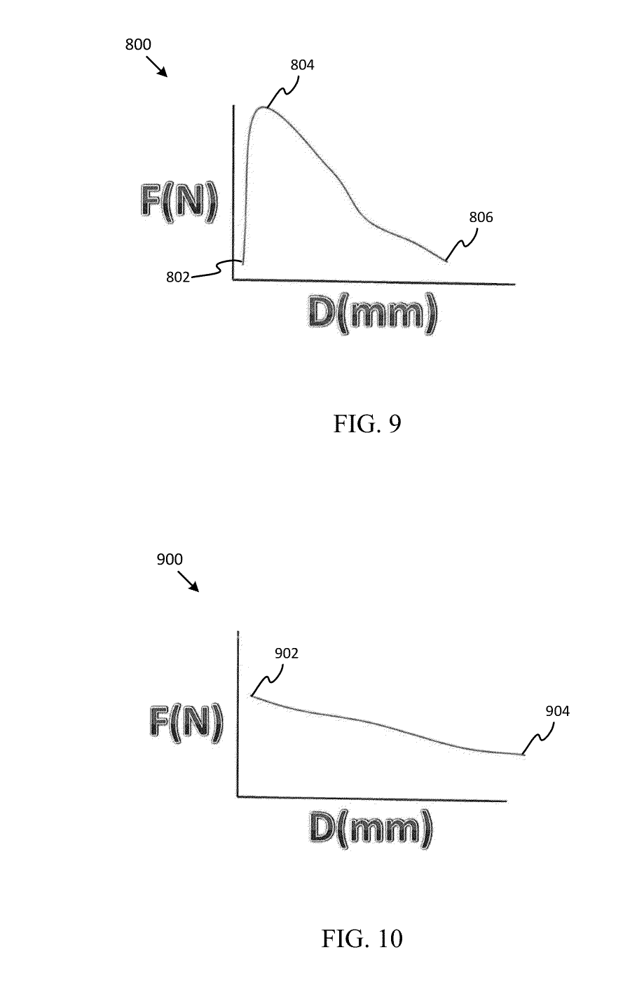

In some embodiments, for example, with reference to FIG. 10, a profile 900 of the applied force, or the force required from the user to generate the required deployment force using a wire collection device as disclosed herein, may decrease from a maximum force 902 to a lower force 904. The difference, or variation, between the maximum force 902 and lower force 904 of the applied force is less than the difference, or variation, between the threshold force 904 and the lower force 906 of the required deployment force. The stent delivery system and wire collection device disclosed herein may reduce the rate of change of the applied force required to generate the required deployment force so as to provide to the user a smoother and more consistent the “touch and feel” during stent deployment.

Although various embodiments of the invention have been described, it will be apparent to those of ordinary skill in the art that many more embodiments and implementations are possible that are within the scope of the invention. Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they are used in a generic and descriptive sense only and not for purposes of limitation unless specifically defined by context, usage, or other explicit designation. It is therefore intended that the foregoing detailed description be regarded as illustrative rather than limiting. And, it should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment. In the event of any inconsistent disclosure or definition from the present application conflicting with any document incorporated by reference, the disclosure or definition herein shall be deemed to prevail.

We claim:

1. A stent deployment system, including a wire collection device comprising:
a profiled retraction wire comprising a distal end coupled to a sledge and a proximal end coupled to a collection spindle, the sledge coupled to a proximal end of an outer sheath comprising a distal end surrounding a stent;
an actuator coupled to the collection spindle and operable to actuate rotation of the collection spindle to collect the profiled retraction wire around the collection spindle, wherein collection of the profiled retraction wire retracts the outer sheath across an outer surface of the stent so as to deploy the stent; and
a varying collection diameter that comprises a diameter of the collection spindle and a combined thickness of collected portions of the profiled retraction wire.

2. The system of claim 1, wherein the actuator comprises a thumbwheel that is offset from the collection spindle and coupled to the collection spindle by a transmission mechanism comprising a belt, a rack and pinion, a clutch, a ratchet, or any combination thereof.

3. The system of claim 1, wherein the actuator comprises a push button rod that forms a rack, the collection spindle forms a pinion.

4. The system of claim 1, wherein the profiled retraction wire comprises a varying diameter that varies along a curvilinear profile.

5. The system of claim 1, wherein the profiled retraction wire comprises a varying diameter that increases from the distal end towards the proximal end of the profiled retraction wire.

6. The system of claim 2, wherein the varying diameter increases linearly from the proximal end towards the distal end of the profiled retraction wire.

7. The system of claim 1, further comprising a diameter increasing construct that increases the collection diameter as the collection spindle rotates so as to increase a retraction speed of the outer sheath.

8. The system of claim 7, wherein the diameter increasing construct comprises a cam that rotates about a central axis of the collection spindle, wherein:
the cam comprises a first end at a minimum distance from an outer surface of the collection spindle and a second end at a maximum distance from the outer surface of the collection spindle;
the cam is rotatable to collect the profiled retraction wire across a surface of the cam extending from the first end to the second end of the cam, thereby increasing the collection diameter as the collection spindle rotates.

9. The system of claim 7, wherein the diameter increasing construct comprises a plurality of beads located along the profiled retraction wire, the plurality of beads aligned to provide support for an inner catheter extending through the outer sheath.

10. The system of claim 9, wherein the plurality of beads comprises grooves to support the inner catheter.

11. The system of claim 9, wherein the plurality of beads increases in size along the profiled retraction wire to accommodate a changing resistance during sheath retraction.

12. The system of claim 9, wherein the plurality of beads comprise rectangular, square, round, or ellipsoid shapes, or any combination thereof.

13. A stent deployment system, including a wire collection device comprising:
a profiled retraction wire coupled to an outer sheath to retract the outer sheath to deploy a stent from a distal end of the outer sheath, the profiled retraction wire comprising a thickness that increases from a proximal end attached to a collection spindle towards a distal end coupled to a proximal end of the outer sheath; and
a thumbwheel coupled to the collection spindle and operable to actuate rotation of the collection spindle to collect the profiled retraction wire around a collection diameter that comprises the diameter of the collection spindle and the thickness of the retraction wire, wherein the collection diameter increases as the profiled retraction wire is collected around the collection spindle.

14. The system of claim 13, further comprising a cam that surrounds the collection spindle such that the cam increases and decreases the collection diameter as the collection spindle rotates to collect the profiled retraction wire around the cam.

15. The system of claim 13, wherein the cam is an eccentric, egg-shaped, or ellipsoid cam.

16. The system of claim 13, wherein the collection spindle comprises a first shoulder and a second shoulder that aligns overlapping layers of the profiled retraction wire.

17. The system of claim 13, wherein the profiled retraction wire comprises linked chains.

18. The system of claim 13, further comprising diameter increasing constructs located along the profiled retraction wire.

19. The system of claim 16, wherein the diameter increasing constructs comprise a plurality of beads.

20. The system of claim 17, wherein the plurality of beads increases in size along the profiled retraction wire.
